(19) United States
(12) Patent Application Publication
(10) Pub. No.: US 2002/0189608 A1
(43) Pub. Date: Dec. 19, 2002

(54) Enhancing athletic performance through the administration of peppermint odor

(76) Inventor: Bryan Raudenbush, St.Clairsville, OH (US)

Correspondence Address: MILLEN, WHITE, ZELANO & BRANIGAN, P.C., ARLINGTON COURTHOUSE PLAZA I, SUITE 1400, 2200 CLARENDON BOULEVARD, ARLINGTON, VA, 22201, US

(21) Appl. No.: 09/825,052

(22) Filed: Apr. 4, 2001

(51) Int. Cl. A61M 11/00

(52) U.S. Cl. 128/200.14

(57) ABSTRACT

A method of improving the strength, endurance, speed and/or perceived workload of an athlete during an athletic performance by administering Peppermint odorant. The peppermint odorant is administered in an amount and manner sufficient to cause olfactory stimulation. Peppermint odorant is infused into training environments, applied directly to an athlete, coated onto surfaces in olfactory proximity to an athlete or impregnated on surfaces in olfactory proximity to an athlete.

### SUMMARY OF THE INVENTION

Peppermint odor administered prior to or during athletic performance improves an athlete's performance.

Thus, this invention relates to a method of improving the athletic performance of a mammal comprising administering to said mammal an olfactory stimulating amount of a Peppermint odorant. Athletic performances which are improved include an increase in speed, an increase in strength, an increase in endurance, a decrease in fatigue and an decrease in perceived workload.

Exemplary activities include swimming, football, karate, lacrosse, hockey, soccer, strength training, weightlifting, shot put, vertical jump, power lifting, conditioning, calisthenics, weight training, cycling, running, sprinting, hurdling, rollerblading, rowing, long jump, boxing, skiing, speed or strength related team sports, anaerobic exercise or aerobic exercise.

Administration can be any route with results in olfactory absorption i.e., stimulation, such as smelling of vapors, ingestion of solids or liquids etc, including but not limited to oral administration, inhalant administration, or administration to mucous membranes. Suitable means of administration include clothing impregnated with peppermint odorant, clothing coated with peppermint odorant, peppermint odor releasing polymer, peppermint odorant impregnated nasal dilators, peppermint odorant coated nasal dilators, peppermint odorant impregnated adhesive strip, peppermint odorant coated adhesive strip, an aerosol peppermint spray, a pump peppermint spray, a nasal peppermint spray, a liquid or solid form of the peppermint odorant contained in a vessel, a liquid or solid form of the peppermint odorant contained in a lozenge, a liquid or solid form of peppermint odor in a gum, a liquid or solid form of peppermint odor in a food product, a peppermint odorant scented mouth guard, a peppermint cream, a peppermint cologne, peppermint odorant impregnated jewelry, peppermint odorant coated jewelry, and a peppermint ointment.

“Athletic performance” is any activity which can be measured by increases or decreases in endurance, strength, speed, energy, perceived workload, fatigue, frustration and/or intensity. “Improvement” in such performance by an individual refers to a measurably better result e.g., higher endurance, strength, speed etc., than for the same individual performing the same activity without the administration of peppermint.

Endurance is the capacity to continue a physical performance over a period of time. An athlete can acquire and build up endurance over time. Energy is the capacity to produce work and is often affected by factors such as mood and health. Duration is the time spent in a single exercise session. Duration, along with frequency and intensity, are factors affecting the effectiveness of exercise.

Anaerobic exercise constitutes short-term activities (usually highly intense) in which muscle fibers derive contractile energy from stored internal compounds without the use of oxygen from the blood. Short bursts of “all-out” effort, such as sprinting or weightlifting are examples of anaerobic activities. Anaerobic activities are activities using muscle groups at high intensities that exceed the body's capacity to use oxygen to supply energy and which create an oxygen debt by using energy produced without oxygen.

Aerobic endurance is the ability to continue aerobic activity over a period of time. Many factors contribute to aerobic strength endurance. Aerobic sports are activities using large muscle groups at moderate intensities that permit the body to use oxygen to supply energy and to maintain a steady state for more than a few minutes. In aerobic exercise oxygen from the blood is required to fuel the energy-producing mechanisms of muscle fibers. Examples are running, cycling and skiing over distance.

Intensity is the rate of performing work or power and can be affected by internal as well as external factors.

Strength is the application of muscular force in any endeavor (speed and distance are not factors of strength). Speed is the rate of motion. There are many different factors that affect strength and speed. Genetic structural and anatomical differences such as the ratio of fast- vs. slow-twitch fibers affect strength and speed. Physiological and biochemical factors such as hormonal function and cardiovascular factors affect strength and speed. Psychoneural and learned responses such as “psych” or arousal levels affect strength and speed. The Psycho neural response would include an athlete's pain tolerance, ability to concentrate, social learning, skill, coordination, mood, motivation and others. External and environmental factors such as equipment, weather and altitude, also effect an athlete's strength and speed.

In a physical therapy setting it is imperative that patients are in an environment conducive to attaining a higher level of motivation, vigor, and mood. A positive mood results in greater muscle relaxation and less muscle contraction. The less a muscle contracts the greater the increase in active range of motion.

Peppermint is a member of the Lamiaceae family. The scientific name is Mentha x piperta. There are numerous common names and varieties of peppermint. Other common names include american mint, brandy mint, lamb mint, lammint. It represents a natural hybrid between spearmint and watermint. Various types of peppermint are cultivated around the world. An intensely spicy, cooling and aromatic oil is extracted from the leaves and the above ground parts of the herb. One of the components of peppermint is menthol, although the distinctive aroma of peppermint is due to not only menthol but other flavenoid components. Peppermint oil commonly appears in lotions, ointments, aromatic mists and aroma therapy formulations. For internal use peppermint oil is a common flavoring agent. If applied undiluted or in high doses, peppermint oil may irritate the skin or mucous membranes, and even at recommended doses peppermint oil can cause a rash or contact dermatitis in sensitive individuals. The FDA has approved peppermint oil as a common cold remedy and it appears in numerous lozenges, decongestants, inhalants, and ointments.

Smell is the sense that enables an organism to perceive and distinguish the odors of various substances. It is also known as olfaction. An olfactory stimulating amount of an odorant is any amount of a particular odorant that will stimulate the nerve fibers that extend from the olfactory cells to the olfactory bulb, i.e., any amount which can be smelled. Most physiologists agree that although a substance must be volatile to be sniffed by the nose, it must subsequently be dissolved in the mucous lining of the nasal cavity to be smelled. Stimulation can be ortho-nasal, i.e., through the nose or retro-nasal, i.e., through the mouth.

Amounts of peppermint which are olfactory stimulating in a given human can be routinely determined in conjunction at most, with a few routine preliminary experiments. These amounts will vary considerably as is well known. For instance, our sense of smell gradually declines beginning when we are in our 30's. Not all decline in smell is age-associated. Some common illnesses also cause a reduction of smell. For example, allergies or nasal infections can reduce the sense of smell. Head injuries and Alzheimer's disease also damage the section of the brain responsible for olfaction and can cause smell reduction or loss. A greater amount of odorant may be used to stimulate the olfactory nerve of an older person or someone suffering from an illness which causes a smell reduction.

Additional factors that could affect the amount of peppermint odorant required would include the air circulation in the environment. If for example, the peppermint odorant was diffused throughout an enclosed space such as a gym, the size of the room would be a consideration in determining the amount of peppermint odorant to use. In a competitive athletic event where one athlete would want the competitive advantages of the peppermint odor without bestowing the advantages on his/her competitors, he/she would administer the peppermint odor in proximity to only their olfactory nerves. Clearly in outdoor events it would be more difficult then in indoor events to scent a large area. Scenting a smaller personal space by keeping the peppermint odor in close proximity to ones olfactory nerves is easier in both situations.

Never the less, there are certain instances when it would be desirable to diffuse peppermint odor into an entire enclosed space. A slight increase in strength, endurance, speed and motivation on a daily basis would greatly affect the long term training efforts of an athlete or team of athletes.

Depending on the desired application, peppermint can be administered in various amounts. Basically any amount of peppermint odor which can be smelled by the athlete or performer will be sufficient.

For use with an oxygen concentrator, delivered via nasal cannula, using an intermittent administration technique of 5 seconds per each 30 second period, the preferred amount is 15 ml of peppermint in a bottle spliced into an oxygen line with an oxygen flow of 1.3 LPM. The oxygen flow rate can be varied, for example from 0.1 LPM to 5.0 LPM, to increase or decease the amount of peppermint odor. For use with an oxygen concentrator, delivered via nasal cannula, in a continuous administration, the preferred administration is 15 ml of peppermint in a bottle spliced into a oxygen line with an oxygen flow of 0.5 LPM. The oxygen flow rate can be varied, for example from 0.1 LPM to 3.0 LPM, to increase or decease the amount of peppermint odor. For use with an oxygen concentrator, delivered via full room odorization, the preferred administration is 15 ml of peppermint in a bottle spliced into an oxygen line with a oxygen flow of 1.0 LPM per 500 cubic feet. The oxygen flow rate can be varied, for example from 0.3 to 5.0 LPM per 500 cubic feet, to increase or decease the amount of peppermint odor.

For application on an absorbent (e.g., tissue, cloth, sponge etc.) device applied under the nose, the preferred administration is from about 0.18 mg/cm² to 30 mg/cm², most preferably about 2.7 mg/cm².

Mammals other than humans, e.g. dogs and horses are well known for having an acute sense of smell. A smaller amount of odorant is needed to stimulate them than would be needed to stimulate a human. Thus, amounts useful in human will be generally useful in other mammals.

Peppermint odor can be administered by many means. Nasal strips, such as the brand name Breathe Right™ product, are about 2 inches long and have a plastic backbone. They are often referred to as nasal dilators. When the strips are bent over the bridge of the nose and the adhesive ends applied to the nostrils, the plastic backbone tends to straighten, lifting the nostrils and dilating the airways. According to one manufacturer, the device decreases nasal airway resistance by 31%. Tens of thousands of professional and non-professional athletes use the Breathe Right™ Nasal Strip in an attempt to augment their performance. The addition of peppermint odor will have an additive or synergistic effect on performance. Useful amounts include about 0.18 mg/cm² to 30 mg/cm². Large nasal strips have also been used on race horses to increase their performance. Nasal strips and other adhesive strips are impregnated or coated with the odor of peppermint and placed on an athlete in proximity to the olfactory nerve cells. Exercise equipment made of peppermint odor releasing polymers can also be used to provide those using the equipment with a continuous release of peppermint odor.

Peppermint odor can also be placed directly on the skin of an athlete below the nose or on the chest. The oil appears to have no strongly adverse reaction with the skin, although as with all essential oils, people with sensitive skin should be careful before they apply it at full strength. It can readily be applied, diluted in a carrier oil, a vegetable oil, a lotion which can be an aqueous suspension, or an ointment, which can often be a petroleum emulsifying ointment base. These aspects are fully conventional. Articles of clothing or jewelry in proximity to the olfactory nerve cells, can also be coated to provide a sustained release of odor.

There are numerous known methods to incorporate aroma into fibers and films used to manufacture any of the foregoing. For example, U.S. Pat. No. 4,713,291 incorporates up to 10% by weight of an aromatic odor into fibers. U.S. Pat. No. 4,950,542 admixes up to 20 parts by weight of an essential oil into a coating mixture. U.S. Pat. No. 3,688,985 provides a plastic article impregnated with a volatile matter. U.S. Pat. No. 3,755,064 deals with filaments containing encapsulated aromatic components. U.S. Pat. No. 4,515,909 concerns resinous compositions for the prolonged release of fragrant substances.

### BRIEF DESCRIPTION OF THE DRAWINGS

FIG. 1: Physical workload measurements: Post-hoc contrasts indicated decreased physical workload in the peppermint condition and increased physical workload in the dimethyl sulfide condition, as compared to the control condition.

FIG. 2: Temporal workload measurements: Post-hoc contrasts indicated decreased temporal workload in the peppermint condition as compared to the control condition.

FIG. 3: Performance measurements: Post-hoc contrasts indicated greater performance in the peppermint condition as compared with the control, dimethyl sulfide and jasmine conditions.

FIG. 4: Required effort measurements: Post-hoc contrasts indicated decreased effort in the peppermint condition as compared to the control condition and the dimethyl sulfide condition.

FIG. 5: Frustration measurements: Post-hoc contrasts indicated decreased frustration in the peppermint condition as compared to the control condition and the dimethyl sulfide condition.

FIG. 6: Vigor measurements: Post-hoc contrasts indicated increased vigor in the peppermint condition as compared to the control condition and the dimethyl sulfide condition.

FIG. 7: Fatigue measurements: Post-hoc contrasts indicated decreased fatigue in the peppermint condition as compared to the control condition and the dimethyl sulfide condition.

In the foregoing and in the following examples, all temperatures are set forth uncorrected in degrees Celsius; and, unless otherwise indicated, all parts and percentages are by weight.

### EXAMPLES

### Example I

### Method

Participants were 40 young adult volunteers (20 males, 20 females, mean age 20 years). They were selected from a variety of inter-collegiate sports teams whose training regime required extensive running (e.g., track, soccer, basketball). Participants received financial compensation following the completion of the experiment.

### Procedures

In the testing phase, participants were asked to perform four tasks: a) dynamometer hand grip (dominant hand), b) 400 m dash (timed), c) pushups to exhaustion (no time limit), and d) 20 basketball free throw shots.

Participants performed the protocol twice, each time under a different odor condition. Condition one consisted of the placement of an odorized (two drops of peppermint oil—Aldrich Co.) adhesive strip under the participant's nose. Condition two was identical, with the exception that the adhesive strip was odor-free. The athletes performed the procedure under both conditions, separated by at least 2 days, with the condition that they received first randomly assigned.

### Results

The data were subjected to a correlated measures t-test for each physical task to determine significance. Means, standard deviations, and effect sizes for the measures are shown in Table 1.

| | | PEPPERMINT | EFFECT SIZE |
|---|---|---|---|
| | NO ODOR | ODOR | (d) |
| 400 m Dash | 81.40 (9.66) | 79.58 (10.31) | 0.47 |
| Free Throws | 8.90 (5.20) | 9.20 (4.13) | 0.09 |
| Hand Grip | 4.07 (0.78) | 4.30 (1.11) | 0.29 |
| Push Ups | 29.45 (12.35) | 31.18 (10.63) | 0.32 |

A significant difference was found between the odorized and non-odorized condition for pushups (t₃₉ = 2.02, p = .051) and 400 m run (t₃₉ = −2.94, p = .005). Differences for the hand grip approached significance (t₃₉ = 1.833, p = .074). No significant differences were noted for the free-throw task (t₃₉ = 0.60, p = .551).
Table 1: Means, Standard Deviations, and Effect Sizes for Tasks Under Different Odor Conditions

### Example II

Athletes performed a modified 15 minute treadmill exercise stress test under each of four odorant conditions (peppermint, jasmine, dimethyl sulfide, or a non-odored control condition) delivered via a nasal cannula. Peppermint odor significantly reduced perceived physical workload, temporal workload, effort and frustration. Self-evaluated performance was also greater in the peppermint condition, and participants rated their level of vigor higher, and their level of fatigue lower. The dimethyl sulfide condition resulted in athletes indicating more fatigue and increased physical work-load.

Participants were 40 young adult volunteers (18 males, 22 females, mean age 20 years). They were selected from a variety of inter-collegiate sports teams at Wheeling Jesuit University whose training regime required extensive running (e.g., track, soccer, basketball). Participants received financial compensation following the completion of the experiment.

Stimuli, Apparatus and Materials

Peppermint (Fragrance Oil—Aldrich, 15 ml), jasmine (Fragrance Oil—Aldrich, 15 ml) or dimethyl sulfide odor (Aldrich, 3 ppm in water) were aerated with low flow oxygen (1.3 LPM) via an oxygen concentrator (AirSep®—Newlife). These odors were chosen based upon past research indicating that they typically effect cognitively based task performance or mood in some way (either enhancing or degrading). Pre-testing with various LPM oxygen settings indicated that odor presentation at 1.3 LPM resulted in a definite perception of the odorant without the athlete noting an overpowering burst of fragrance.

Participants also completed the NASA-TLX (Task Load Index; Hart & Staveland, 1988), which is a subjective scale of mental and physical workload. The TLX is a multi-dimensional scale which measures specific components of workload in a given task along three dimensions related to demands imposed on the participant by the task (mental, physical, temporal), and three dimensions related to the interaction of the participant and the task (effort, frustration, performance). Rating scale definitions are found in Table 1.

TABLE 1
RATING SCALE DEFINITIONS

| COMPONENT | DESCRIPTIONS |
|---|---|
| MENTAL | How much mental and perceptual activity was required |
| DEMAND | (e.g., thinking, deciding, calculating, remembering, |
| | looking, searching, etc.)? Was the task easy or |
| | demanding, simple or complex, exacting or forgiving? |
| PHYSICAL | How much physical activity was required? Was the |
| DEMAND | task easy or demanding, slow or brisk, slack or |
| | strenuous, restful or laborious? |
| TEMPORAL | How much time pressure did you feel due to the rate |
| DEMAND | or pace at which the tasks or task elements occurred? |
| | Was the pace slow and leisurely or rapid and frantic? |
| EFFORT | How hard did you have to work (mentally and |
| | physically) to accomplish your level of performance? |
| PERFORMANCE | How successful do you think you were in accomplish- |
| | ing the goals of the task set by the experimenter |
| | (or yourself)? How satisfied were you with your |
| | performance in accomplishing these goals? |
| FRUSTRATION | How insecure, discouraged, irritated, stressed and |
| LEVEL | annoyed versus secure, gratified, content, relaxed and |
| | complacent did you feel during the task? |

Based on the recommended measurement procedure of Hart and Staveland (1988), participants were presented with a 12 cm line with the endpoints of the line marked “low” and “high” for each of the six aspects of workload. They were then asked to place a hash mark on the line to indicate their level of workload. The distance of their hash mark from the left (beginning) of the 12 cm line was measured in millimeters, and that number was taken as a numeric indication of workload.

The final assessment inventory was the Profile of Mood States. (POMS; McNair, Lorr & Droppleman, 1971). The POMS contains a list of 65 adjectives concerning mood. Subjects indicate the extent to which each adjective describes them at a particular moment using a 5 point scale. For the present investigation, questions were assessed within two sub-scales: fatigue and vigor.

### Procedure

Participants were advised that they should not eat within 3 hours of the testing conditions. Upon entering the testing facility, they were connected to a vital signs monitor (Keller KMS-890+), and baseline measures of oxygen saturation, pulse, blood pressure and MAP were taken. They began the testing under a modified version of the Bruce Protocol (Bruce, 1967), which is by far the most commonly used protocol in treadmill exercise or stress testing and approved by the American Heart Association (AHA, 1997). To minimize any effects of individual differences due to walking/running style, subjects were instructed to walk erect (not bent over), near the front of the belt (to allow for normal strides), and to not use the hand rail for support unless absolutely necessary. These instructions were repeated each time the participants began a testing session, and in the event that they began to deviate (e.g., moved too close to the front of the belt, attempted to use the hand rails) they were reminded of the protocol.

Subjects began walking on the treadmill (Cybex Trotter 700T) at 1.7 mph on a 7% grade and progressed to the point of 5 mph on a 15% grade. Table 2 depicts the progression from the beginning to the end of the test. As can be seen, the incline and speed are both increased every 3 minutes until the subject has performed 15 minutes of activity. Participants continued on the treadmill until they had performed 15 minutes of testing, and progressed through the 5 stages of the Bruce Protocol. Research indicates that by the completion of the 5 stages, most, if not all, of the subjects should have reached their target heart rate, thus making this procedure quite adequate in promoting a progressive level of difficulty (Bruce, 1963).

TABLE 2
Modified Bruce Protocol Testing Stages of Progression

| | SPEED | | TIME | CUMULATIVE TIME |
|---|---|---|---|---|
| STAGE | (mph) | GRADE | (min) | (min) |
| 1 | 1.7 | 7% | 3 | 3 |
| 2 | 2.5 | 9% | 3 | 6 |
| 3 | 3.4 | 11% | 3 | 9 |
| 4 | 4.2 | 13% | 3 | 12 |
| 5 | 5.0 | 15% | 3 | 15 |

Participants performed the protocol four times, each time under a different odor condition, separated by at least 48 hours. In addition, the four testing times for each participant were scheduled such that they would occur within plus or minus one hour of the same time of day (within a 3 hour block), thus reducing within-subject variability that would be due to differences in testing times. During one of the conditions, they completed the test with normal un-odored low-flow oxygen (1.3 LPM) being presented to them through a nasal cannula. The cannula is the type that is used to deliver oxygen in medical settings. In the other conditions, they completed the protocol with the peppermint-, jasmine- or dimethyl sulfide-odored low-flow oxygen (1.3 LPM) being presented to them through a nasal cannula. The order of the conditions they received was randomly assigned. In the odor condition, odor was presented for 5 seconds every 1 minute, beginning 30 seconds after the initiation of the test and continuing until 14 minutes and 30 seconds into the test. For the remainder of the odor condition time, un-odored low-flow oxygen was presented. In the no-odor condition, only un-odored low-flow (1.3 LPM) oxygen was presented.

At the end of each condition, participants completed the NASA-TLX questionnaire and the vigor and fatigue sub-scales of the POMS, during which post-condition physiological measurements were taken.

### Results—Physiological Measures

Measures of oxygen saturation, pulse, blood pressure and MAP were calculated before and after each odorant condition. For each physiological measure, the difference was taken between these pre and post recordings as an indication of the change in these variables during the experimental session. Mean differences between the pre and post conditions and standard deviations can be found in Table 3. A negative number indicates that the post condition measure was greater than the pre-condition measure.

TABLE 3
Physiological Measurement Differences (pre vs. post) and
Standard Deviations for the Four Odorant Conditions

| | | DIMETHYL | | |
|---|---|---|---|---|
| VARIABLE | CONTROL | SULFIDE | JASMINE | PEPPERMINT |
| O₂ | 1.10 | 1.00 | 0.68 | 0.97 |
| | (1.54) | (2.94) | (2.30) | (2.20) |
| PULSE | −37.46 | −41.13 | −36.83 | −41.12 |
| | (19.17) | (19.81) | (19.47) | (19.71) |
| SYSTOLIC | −45.74 | −38.20 | −41.48 | −41.23 |
| | (22.83) | (17.96) | (23.25) | (19.87) |
| DIASTOLIC | −9.26 | −11.63 | −9.36 | −7.70 |
| | (11.05) | (17.44) | (17.97) | (12.79) |
| MAP | −19.54 | −19.42 | −16.38 | −20.24 |
| | (11.92) | (12.730 | (13.10) | (12.44) |

One-between (gender), one-within (odor condition) ANOVAs were performed to assess whether the pre-post changes were significant for any of the physiological variables among the odor conditions.

Oxygen Saturation

There was no effect for odor condition (F=0.32, p>0.05), although there was a significant gender effect (F=6.3, p<0.05). A greater pre-post change was noted for females (M=2.00, SE=0.42) than males (M=0.50, SE=0.42). The odor condition x gender interaction was not significant (F)=0.32, p>0.05.

Pulse

There was no effect for odor condition (F=0.64, p>0.05), gender (F=1.25, p>0.05), or the odor condition x gender interaction (F=2.51, p>0.05)

Systolic Blood Pressure

There was no effect for odor condition (F=2.27, p>0.05), gender (F=0.05, p>0.05), or the odor condition x gender interaction (F=0.52, p>0.05).

Diastolic Blood Pressure

There was no effect for odor condition (F=0.56, p>0.05), although there was a significant gender effect (F=7.61, p<0.01). A greater pre-post change was noted for females (M=−14.33, SE=2.06) than males (M=−6.07, SE=2.17). The odor condition x gender interaction was not significant (F=0.25, p>0.05).

MAP

There was no effect for odor condition (F=0.18, p>0.05), although there was a significant gender effect (F=14.25, p<0.01). A greater pre-post change was noted for females (M=−23.44, SE=1.73) than males (M=−13.92, SE=1.84). The odor condition x gender interaction was not significant (F=0.61, p>0.05).

### Workload Measures

Means and standard deviations for the six measures of workload can be found in Table 4. One-between (gender), one-within (odor condition) ANOVAs were performed to assess whether workload differences were significant among the odor conditions.

TABLE 4
NASA-TLX Workload Measurement Means and
Standard Deviations for the Four Odorant Conditions

| WORKLOAD | | DIMETHYL | | PEPPER- |
|---|---|---|---|---|
| COMPONENT | CONTROL | SULFIDE | JASMINE | MINT |
| MENTAL | 39.76 | 43.95 | 42.40 | 36.32 |
| | (26.25) | (25.91) | (26.49) | (25.13) |
| PHYSICAL | 65.46 | 72.21 | 67.19 | 60.34 |
| | (28.58) | (22.03) | (27.66) | (26.69) |
| TEMPORAL | 51.83 | 49.71 | 50.02 | 42.61 |
| | (24.04) | (22.53) | (24.99) | (25.79) |
| PERFORMANCE | 74.22 | 73.60 | 74.29 | 81.46 |
| | (21.12) | (22.56) | (25.77) | (17.33) |
| EFFORT | 63.63 | 66.93 | 61.74 | 56.83 |
| | (26.07) | (24.08) | (28.08) | (26.40) |
| FRUSTRATION | 35.71 | 35.74 | 33.21 | 27.37 |
| | (24.34) | (22.01) | (23.34) | (21.49) |

Mental Workload

No significant differences were found for odor condition (F=2.06, p>0.05), gender (F=1.03, p>0.05) or the odor condition x gender interaction (F=0.12, p>0.05).

Physical Workload

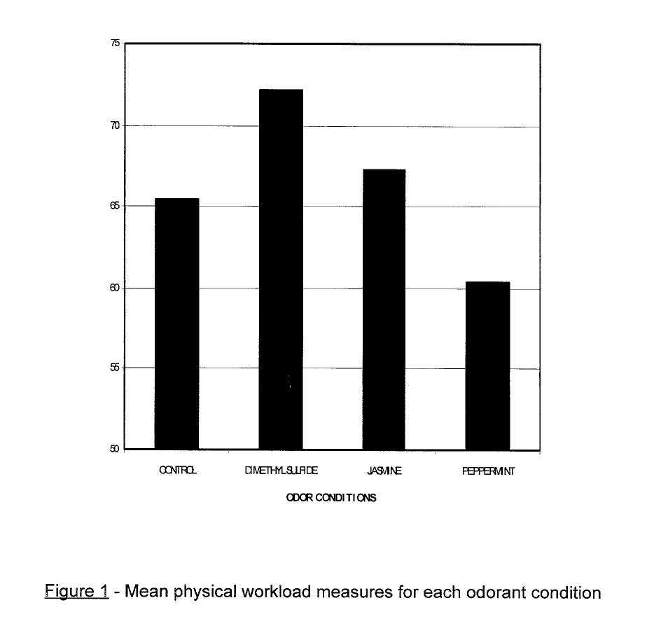

A significant difference was found for odor condition (F=4.31, p<0.01). Post-hoc contrasts indicated decreased physical workload in the peppermint condition and increased physical workload in the dimethyl sulfide condition, as compared to the control condition (see FIG. 1). Physical workload was also lower in the peppermint condition as compared to the dimethyl sulfide condition. No differences were found for gender (F=0.10, p>0.05) or the odor condition x gender interaction (F=1.30, p>0.05).

Temporal Workload

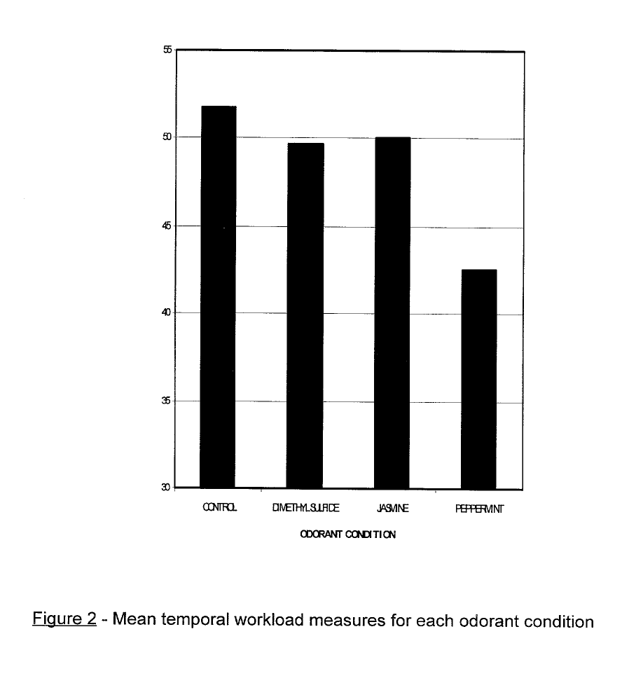

A significant difference was found for odor condition (F=2.88, p<0.05). Post-hoc contrasts indicated decreased temporal workload in the peppermint condition as compared to the control condition (see FIG. 2). No differences were found for gender (F=0.10, p>0.05) or the odor condition x gender interaction (F=0.65, p>0.05).

Performance

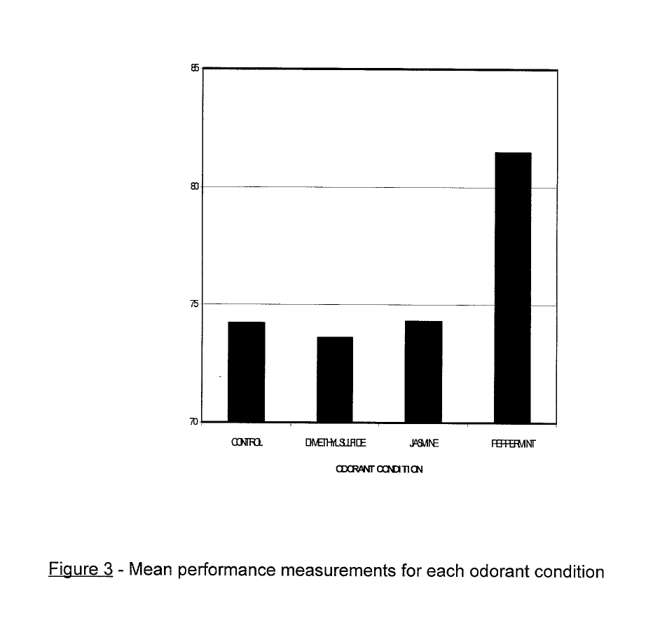

A significant difference was found for odor condition (F=2.80, p<0.05). Post-hoc contrasts indicated greater performance in the peppermint condition as compared with the control, dimethyl sulfide and jasmine conditions (see FIG. 3). No differences were found for gender (F=2.36, p>0.05) or the odor condition x gender interaction (F=1.04, p>0.05).

Effort

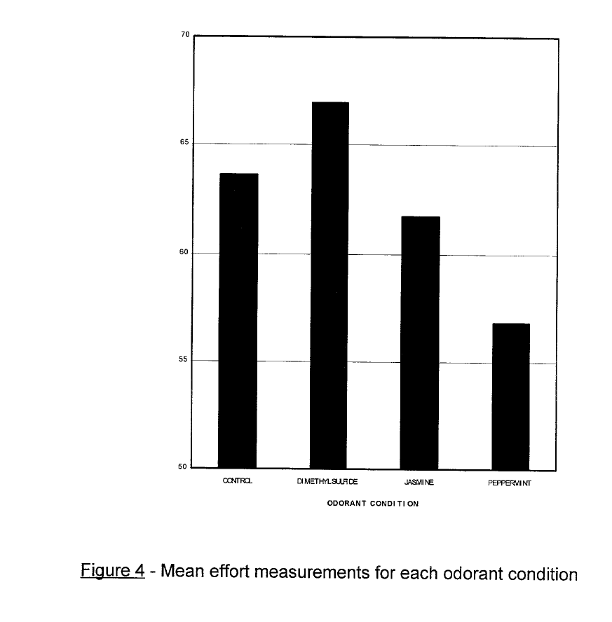

A significant difference was found for odor condition (F=2.86, p<0.05). Post-hoc contrasts indicated decreased effort in the peppermint condition as compared to the control condition and the dimethyl sulfide condition (see FIG. 4). No differences were found for gender (F=0.02, p>0.05) or the odor condition x gender interaction (F=1.42, p>0.05).

Frustration

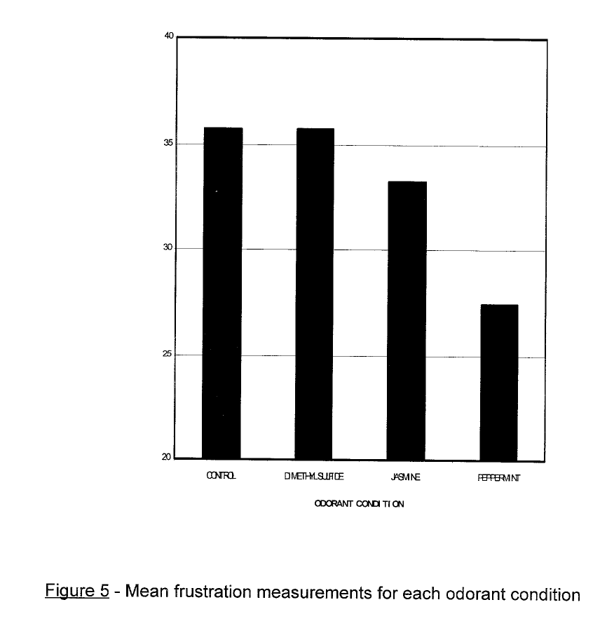

A significant difference was found for odor condition (F=2.79, p<0.05). Post-hoc contrasts indicated decreased frustration in the peppermint condition as compared to the control condition and the dimethyl sulfide condition (see FIG. 5). No differences were found for gender (F=2.03, p>0.05) or the odor condition x gender interaction (F=0.63, p>0.05).

### Mood Measures

Means and standard deviations for the POMS sub-scales of vigor and fatigue are shown in Table 5. One-between (gender), one-within (odor condition) ANOVAs were performed to assess whether differences in mood were significant among the odor conditions.

TABLE 5
POMS Subscale Means and Standard Deviations
for the Four Odorant Conditions

| | | DIMETHYL | | |
|---|---|---|---|---|
| MOOD | CONTROL | SULFIDE | JASMINE | PEPPERMINT |
| VIGOR | 17.27 | 17.78 | 17.83 | 19.83 |
| | (5.81) | (5.94) | (5.46) | (5.93) |
| FATIGUE | 8.12 | 8.95 | 7.83 | 6.49 |
| | (5.60) | (5.46) | (5.70) | (4.36) |

Vigor

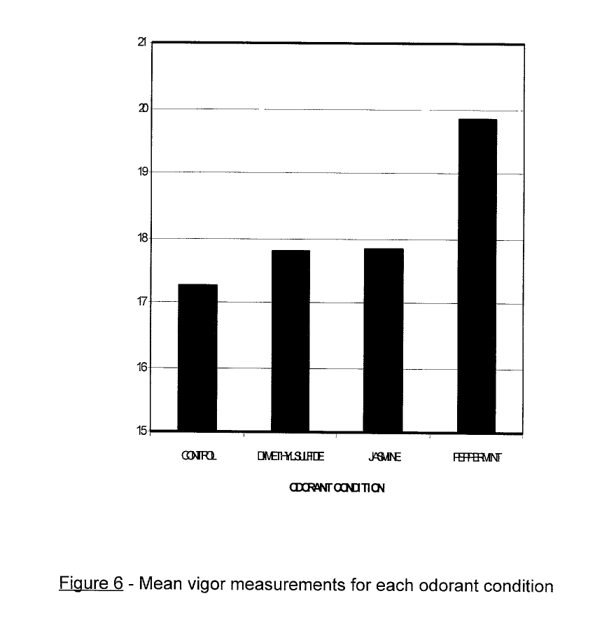

A significant difference was found for odor condition (F=3.27, p<0.05). Post-hoc contrasts indicated increased vigor in the peppermint condition as compared to the control condition and the dimethyl sulfide condition (see FIG. 6). No differences were found for gender (F=0.44, p>0.05) or the odor condition x gender interaction (F=0.39, p>0.05).

Fatigue

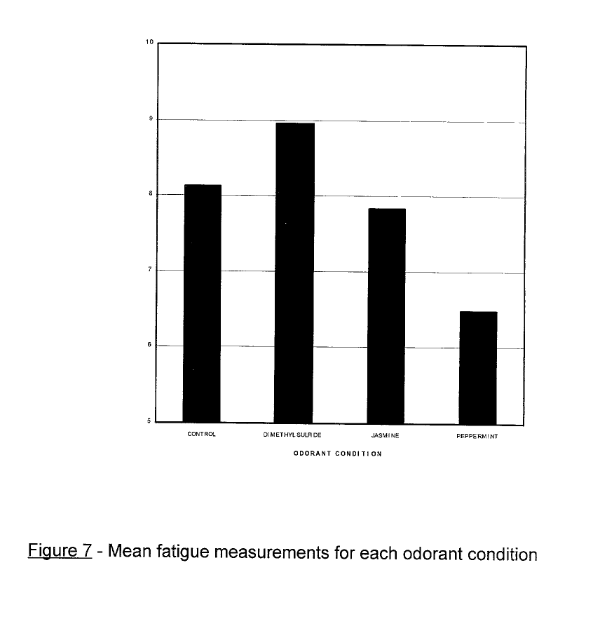

A significant difference was found for odor condition (F=3.82, p<0.05). Post-hoc contrasts indicated decreased fatigue in the peppermint condition as compared to the control condition and the dimethyl sulfide condition (see FIG. 7). No differences were found for gender (F=0.66, p>0.05) or the odor condition x gender interaction (F=0.26, p>0.05).

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

What is claimed is:

1. A method of improving the athletic performance of a mammal comprising administering to said mammal an olfactory stimulating amount of a peppermint odorant.

2. A method according to claim 1, wherein the improvement comprises an increase in speed.

3. A method according to claim 1, wherein the improvement comprises an increase in strength.

4. A method according to claim 1, wherein the improvement comprises an increase in endurance.

5. A method according to claim 1, wherein the improvement comprises a decrease in fatigue.

6. A method according to claim 1, wherein the improvement comprises a decrease in perceived workload.

7. A method according to claim 1, wherein the athletic performance is anaerobic.

8. A method according to claim 1, wherein the athletic performance is aerobic.

9. A method according to claim 1, wherein the athletic performance is weightlifting, shot put, vertical jump, power lifting, conditioning, calisthenics, weight training, cycling, running, sprinting, hurdling, rollerblading, rowing, long jump, boxing, skiing, swimming, football, karate, strength training, lacrosse, hockey, soccer, speed or strength related team sports, anaerobic exercise or aerobic exercise.

10. A method according to claim 1, wherein the administration is ortho-nasal inhalation of odor.

11. A method according to claim 1, wherein the administration is retro-nasal.

12. A method according to claim 1, wherein the administration is through the mucus membranes.

13. A method according to claim 1, wherein the odorant is administered in a form selected from peppermint impregnated or coated mouth-guards, peppermint impregnated or coated face-guards, clothing impregnated with peppermint odorant, clothing coated with peppermint odorant or a peppermint odor releasing polymer.

14. A method according to claim 1, wherein the odorant is administered in a form selected from peppermint odorant impregnated nasal dilators, peppermint odorant coated nasal dilators, peppermint odorant impregnated adhesive strip or peppermint odorant coated adhesive strip.

15. A method according to claim 1, wherein the odorant is administered in a form selected from an aerosol spray, a pump spray, a nasal spray, a liquid or solid form of the peppermint odorant contained in a vessel, a liquid or solid form of the peppermint odorant contained in a lozenge, a liquid of solid form of the peppermint odorant in a gum, a liquid or solid form of the peppermint odorant in a food product, a cream, a cologne, peppermint odorant impregnated jewelry, peppermint odorant coated jewelry, and ointment.

16. A method according to claim 1, wherein the mammal is a human.

17. A method according to claim 1, wherein the mammal is a horse or dog.
